(19) United States
(12) Patent Application Publication
(10) Pub. No.: US 2023/0145152 A1
(43) Pub. Date: May 11, 2023

(54) A LYOPHILIZED TEST KIT FOR DETERMINATION OF ANTIMICROBIAL BIOLOGICAL ACTIVITY OF PHAGE PREPARATIONS AGAINST THE BACTERIA STAPHYLOCOCCUS AUREUS

(71) Applicant: MB Pharma s.r.o., Praha 2 (CZ)

(72) Inventors: Martin Benesik, Strani (CZ); Marek MOSA, Meceriz (CZ)

(73) Assignee: MB Pharma s.r.o., Praha 2 (CZ)

(21) Appl. No.: 17/915,625

(22) PCT Filed: Mar. 16, 2021
(86) PCT No.: PCT/CZ2021/050032

§ 371 Date: Sep. 29, 2022

(30) Foreign Application Priority Data
Mar. 31, 2020 (CZ) PV 2020-181

(51) Int. Cl. C12Q 1/18; C12Q 1/70; C12N 7/00

(52) U.S. Cl. CPC C12Q 1/18; C12Q 1/70; C12N 7/00; C12N 2795/00021

(57) ABSTRACT

A lyophilized test kit for determination of biological antimicrobial effectiveness of a phage lysate on the basis of a microtitration plate wherein individual wells of one series contain at least 50 μl of a lysate of one of the following phages or their combination: MB 401, MB 402, MB 403, MB SA2, MB SA3, MB SA5, namely in the ten-fold dilution at the concentrations of 1×10⁹-1×10².

### CROSS-REFERENCE TO RELATED APPLICATIONS

This application is a National Phase Application of PCT International Application No. PCT/CZ2021/050032, filed on Mar. 16, 2021, which claims priority to Czech Republic Patent Application No. PV 2020-181, filed on Mar. 31, 2020, which are hereby incorporated by reference in their entirety.

### FIELD OF THE INVENTION

The technical solution relates to a test kit comprising a lyophilized phage lysate acting against the bacteria Staphylococcus aureus. The kit is suitable for very sensitive testing of antimicrobial biological activity of the phage lysate against the pathogenic bacteria S. aureus directly on users' premises.

### BACKGROUND OF THE INVENTION

Bacteria of the genus Staphylococcus are a frequent causative agent of human as well as animal diseases. In the recent years, they have represented a big problem in human medicine as well as the veterinary environment. According to the World Health Organization (WHO), 33,000 people die every year in association with bacteria resistant to antibiotics in the EC countries. According to current WHO estimates, by 2050, more people will die from infection caused by antibiotic resistant bacteria than from cancer diseases. Within the frame of solutions to the antibiotic crisis, new antimicrobial agents, as well as completely novel approach to treatment and diagnostics of bacterial causative agents of diseases (Duval et al., 2019) have to be sought.

Besides other possible antimicrobial agents, one option is represented by bacteriophages and their use against bacteria. The so-called phage therapy is no new method. Currently it is increasingly getting into the focus of physicians and scientific teams, in the very association with the antibiotic resistance of bacteria (Lin et al., 2017). The principle of phage therapy was described a very long time ago. However, only the development of modern microbiological and molecular biological methods, describing both phages and pathogenic bacteria can safely and quickly introduce phage therapy into practice. The approach to the treatment of bacterial infections by means of phages diametrically differs from the common antibiotic treatment. While antibiotics are often broad-spectrum and act across bacterial genera and species, phages are usually very specific and act upon a particular bacterial genus, species, and possibly only upon specific bacterial strains. While antibiotics can be applied without detailed knowledge of the bacterial causative agent of the disease, phage treatment requires diagnostics of bacteria. In antibiotic treatment, detailed diagnostics is only applied if strong antibiotics are not effective, and it should be established whether there is an antibiotic to which the bacteria are sensitive. With phages and phage mixes, such diagnostics is necessary immediately before the initiation of the phage therapy. Since the action mechanisms of phages and related bacterial diagnostics differ from the common treatment procedure, quick diagnostic methods need to be adapted to the phage therapy needs.

There are a number of pharmacopeial methods for testing the activity of antimicrobial agents that are already routinely used in microbiological laboratories. These include a diffusion method where an antibiotic is released from a disk to the surroundings and eliminates the growth of bacteria in the presence of the antibiotic. The activity is then assessed based on the size of the inhibition area around the disk. In the case of resistance of the bacteria to the antibiotic, there is no inhibitory zone. However, this method is not well applicable to bacteriophages as they are much larger than antibiotics, and therefore they do not diffuse so quickly to the surroundings.

Another method is the Minimum Inhibitory Concentration (MIC) where the concentration of an antibiotic inhibiting visible bacterial growth is determined (Andrews, 2002). It is assessed by a mere visual check, and no spectrophotometer is required. Here, the minimum concentration of the antibiotic that inhibits growth of the bacteria is determined. For the test, the concentration (CFU) of the bacteria must be clearly defined. On a similar basis, the Minimum Bactericidal Concentration (MBC) is determined, which is a concentration that will prevent bacterial growth in a medium. For these methods, procedures have been defined by the State Authority, which are used by diagnostic facilities in hospitals and other institutions. However, there are no officially approved methods of determination of antimicrobial biological activity of phage lysates, and therefore the testing results in individual laboratories may considerably differ. A method that perhaps approximates the MIC determination of antibiotics is the Appelman Test used since 1921 (Merabishvili et al., 2014), which is however different from the MIC determination, so the results of both the tests cannot be related to each other. In addition, testing the biological activity of phages is mostly the matter of several specialized facilities which work with phages. Thus, the process from the isolation of the pathogenic bacteria, transport to a specialized facility, and the entire feedback to the patient takes a very long time as compared to testing the biological activity directly in the facility where the patient is present.

There are several methods of determining the activity of bacteriophages against particular bacteria. It is the plaque method when individual dilutions of the phage are dripped onto a growing bacterial culture in double-layered agar, and based on the formation of bacteriophage plaques, the concentration of bacteriophages, i.e., the plaque forming unit (PFU), is determined (Anderson et al., 2011). Phages may be dripped on the culture in the respective dilution, or they are directly added to agar to the bacterial culture. Thus, based on the number of plaques and the respective dilution, the concentration of viable phages contained in 1 ml of lysate is calculated. Sometimes, the numbers may differ due to the used culture media, titration bacteria, etc. This method provides a result that determines the number of viable phages, but it only provides partial information about the rate of antimicrobial biological activity of the phage against the bacteria. Only a comparison of dripping the phage on more strains makes it possible to judge how active the phage is. However, this method poses high material demands, comprises more steps that are prone to mistakes, and must be optimized in every facility. Other methods as qPCR etc. can also be used to determine the number of bacteria, but they do not provide information on how many phages are able to proliferate on the host bacteria (Anderson et al., 2011).

Companies, laboratories, and collections of microorganisms often own very specific phages, and their skilled personnel know methods for the determination of the sensitivity of bacteria. In such a case, the patient or medical facility must collect pathogenic, often resistant bacteria, and send them for testing. As the specific phages stored in a single source may not work, the bacteria should suitably be sent for testing to more locations. This makes quick and efficient diagnostics of sensitivity of bacteria to phages impossible. With regard to high dynamics of development of resistance of bacteria to antibiotics, a quick methodology of determining sensitivity of bacteria to other antimicrobial agents, which may also be bacteriophages, is necessary. Such a methodology is absent in diagnostic laboratories for the time being and no phages are available for testing with a method that could be routinely used just in diagnostic laboratories or directly where pathogenic bacteria have been isolated. This may be solved by good training of staff or with a suitable test kit that may be used anywhere.

Bacteriophages, similarly to other biological material are often not long term stable in a liquid form. For this reason, other options of extending the shelf life, i.e., usability period, of phage preparations, or as in this case, a test kit containing phages, must be looked for. One of the options of enhancing stability of phages is lyophilization, which generally prevents degradation of proteins and biological materials, thus extending stability.

### DISCLOSURE OF THE INVENTION

The above-mentioned shortcomings are eliminated with a test of biological effectiveness of phages acting against the bacteria Staphylococcus aureus (S. aureus). Lyophilized phages are contained in a microtitration plate in various concentrations. Growth media, a buffer or water are added into wells with lyophilizate at the volume of 50-100 μl, and bacteria at an exactly predefined concentration are added to these concentration series using a similar procedure that is described for the MIC determination application, representing a routine for diagnostic laboratories.

### BRIEF DESCRIPTION OF THE DRAWINGS

FIG. 1 is an example of dilutions of phage lysates in wells of one series of a microtitration plate.

FIG. 2 is an example of a microtitration plate comprising 6 strains of phages (Table 1) in various dilutions.

FIG. 3 are examples of results of a test of biological effectiveness of phage lysates. In the case of unclear results (may be resistant as well as sensitive), additional testing by dripping is suitable.

### DESCRIPTION OF EMBODIMENTS OF THE INVENTION

A 96-well microtitration plate can be prepared wherein in each numbered series, there will be from A to H phage dilutions from 10⁹ (well A) to 10² (well H). These concentrations may vary, but they may always be lower by one order at the most. This decrease may be caused by a titer loss during lyophilization. However, each batch of the kit will be tested for titer decrease and the exact titer value will be declared for each batch. The test kit will also comprise a test tube with lyophilized bacteria that will be sensitive to the phage/phages contained in the test kit. This bacterium will be prepared and tested in the same way as the clinically tested isolate and will serve as a positive control. Plates with lyophilized phage lysates can be stored at a temperature from 0° C. to 25° C. In case of unsuitable storage, the activity may be affected due to phage degradation.

The mixtures of the phage and bacteria are then cultivated at the temperature of 37° C. for 15 to 20 hours and then the results are evaluated similarly to MIC. If the bacteria are sensitive to the phage, no turbidity of the sample occurs up to the dilution of −4. If the bacteria are resistant to the phage, all or most of the wells will remain turbid. In an undiluted sample, no turbidity often occurs, which is caused by the fact that at a high concentration (on the order of 10⁹), the phage inhibits growth although it is not able to proliferate on the bacteria. Only if the sample is clear at a concentration on the order of 10⁷ (dilution to −3), a definite conclusion can be drawn that the bacteria are sensitive to the phage. The results of this method can then be compared to the effect of antibiotics as they were determined using a similar methodology and can be related to each other.

The basis for the preparation of the test kit is a phage lysate acting against the bacteria S. aureus, namely in particular the phages MB401, MB402, MB403, MBSA2, MBSA3, MBSA5 (Table 1) at the concentration (titer) of 1×10⁹-1×10¹⁰. The lysate is then diluted as follows in a ten-fold dilution and divided in the particular quantity into one row of the microtitration plate.

TABLE 1
Bacteriophages used in the test kit for determination
of biological effectiveness of phage lysates. “% G +
C” defines the contents of guanine and cytosine.

| Bacteriophage | Family | % G + C | Genome size [kbp] |
|---|---|---|---|
| MB401 | Myoviridae | 30 | 179 |
| MB402 | Myoviridae | 30.3 | 140 |
| MB403 | Myoviridae | 30.2 | 142 |
| MBSA2 | Podoviridae | 29.1 | 18 |
| MBSA3 | Myoviridae | 30.3 | 146 |
| MBSA5 | Myoviridae | 30.3 | 146 |

Preparation of Stock Lysates:

1. Dose 50 ml of the phage lysate at a minimum concentration of 1×10⁹-1×10¹⁰ into the sterile 50-ml test tube number 1.

2. Aseptically dose 45 ml of liquid sterile SA media (Trypton 5 g/l, Yeast extract 2 g/l, NaCl 5 g/l) into another 7 test tubes (numbers 2-8).

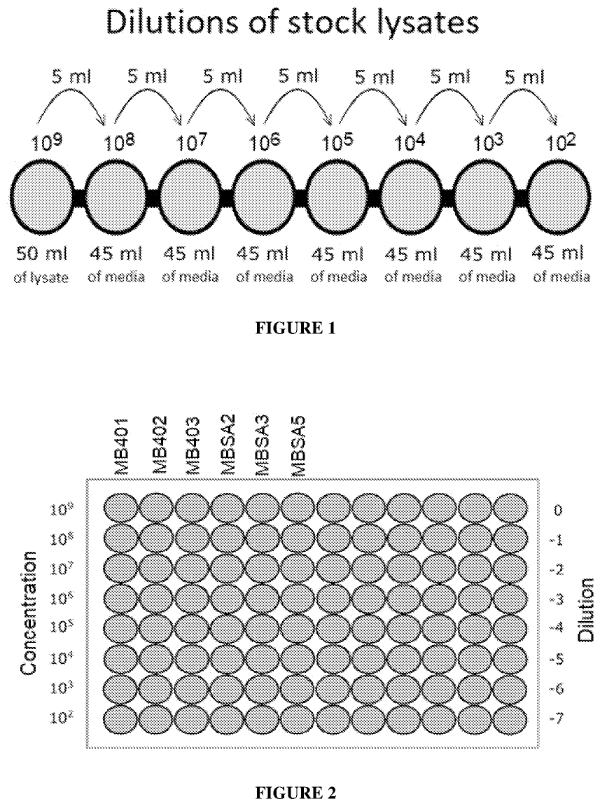

3. Aseptically transfer the exact volume of 5 ml of the lysate from the test tube no. 1 to the test tube no. 2 and stir the test tube no. 2 thoroughly (FIG. 1).

4. Aseptically transfer the exact volume of 5 ml of the lysate from the test tube no. 2 to the test tube no. 3 and stir the test tube no. 3 thoroughly. Proceed in the same way until the test tube no. 8. Finally, there are 45 ml of the lysate in the test tubes 1-7 and the test tube no. 8 contains 50 ml of the most diluted lysate.

Preparation of a Plate from the Stock Lysates:

One series of wells of the microtitration plate always comprise one phage strain at dilutions differing by one order on the consecutive orders of 10⁹ to 10² in the decreasing order from the highest to the lowest dilution (from well A in the plate to well H).

1. From a 50-ml test tube comprising phages at the concentration of 1×10⁹, transfer 100 μl of lysate into well A in the plate. Transfer another 100 μl of the lysate of the lower dilution of the ten-fold series (1×10⁸) to the next well in the series no. 1.

2. Proceed in a similar way until you fill all the wells in the series (column) no. 1 in the microtitration plate (FIG. 2).

3. In a similar way, in the microtitration plate, the other series (2-12) can be prepared wherein 12 different phages may be contained or 12 samples of bacteria may be tested (FIG. 2).

4. Place the microtitration plate in a freeze-dryer and after the completion of the lyophilization, determine the titer in individual rows in randomly selected plates of one batch.

Two types of the bacteria (sensitive 1137 and less sensitive—resistant SA A 4609 FNO) S. aureus were inoculated into 10 ml of the media and cultivated at 37° C. for 18-24 hours. An inoculum for testing of biological effectiveness of the phages is prepared from the culture grown this way. The night culture is diluted to the turbidity value of 1 McFarland and 2 ml of this dilution are supplemented to 20 ml with the media/physiological solution. This suspension is poured onto a sterile Petri dish. Sterile tips of a multi-channel pipette are dipped into the suspension and after the dipping, they are transferred and wetted in the micro-tubes of a strip or in the dissolved lyophilizate in the microtitration plate. It is cultivated at 37° C. for 12-18 hours, ideally under gently shaking. The result is then evaluated based on the turbidity in individual wells of the plate (Table 2). The results show a clear difference in sensitivity of the bacteria S. aureus 1137 and the less sensitive/resistant strain SA A 4609 FNO.

TABLE 2
Testing sensitive and resistant bacteria with dilutions
of the phages MB401, MB402, MB403, and MBSA2. The
numbers identify dilutions, K- is bacteria growth
control and KS is control of sterility of work and
lysate in the strips. Lysis is marked L, very moderate
turbidity is marked VMT, and turbidity is marked T.

| Dilutions | | | | | | | | | |
|---|---|---|---|---|---|---|---|---|---|
| −9 | −8 | −7 | −6 | −5 | −4 | −3 | −2 | −1 | 0 |

Testing Sensitive Bacteria

| | | | | | | | | | | | | |
|---|---|---|---|---|---|---|---|---|---|---|---|---|
| MB401 | K- | KS | T | T | T | T | VMT | L | L | L | L | L |
| MB402 | K- | KS | T | T | T | T | VMT | L | L | L | L | L |
| MB403 | K- | KS | T | T | T | T | VMT | L | L | L | L | L |
| MBSA2 | K- | KS | T | T | T | T | VMT | L | L | L | L | L |

Testing Moderately Resistant Bacteria

| | | | | | | | | | | | | |
|---|---|---|---|---|---|---|---|---|---|---|---|---|
| MB401 | K- | KS | T | T | T | T | T | T | T | VMT | L | L |
| MB402 | K- | KS | T | T | T | T | T | T | T | VMT | L | L |
| MB403 | K- | KS | T | T | T | T | T | T | T | T | L | L |
| MBSA2 | K- | KS | T | T | T | T | T | T | T | VMT | L | L |

The strains of S. aureus bacteriophage with working identification MB401, MB402, MB403, MBSA2, MBSA3, and MBSA5 were deposited at the Leibniz Institute DSMZ, German Collection of Microorganisms and Cell Cultures GmbH, InhoffenstraBe 7B, 38124 Braunschweig, Germany on Mar. 19, 2020 under the conditions of the Budapest Treaty and were assigned accession numbers DSM 33472, DSM 33473, DSM 33474, DSM 33475, DSM 33476, and DSM 33477, respectively.

### EXAMPLES

### Example 1

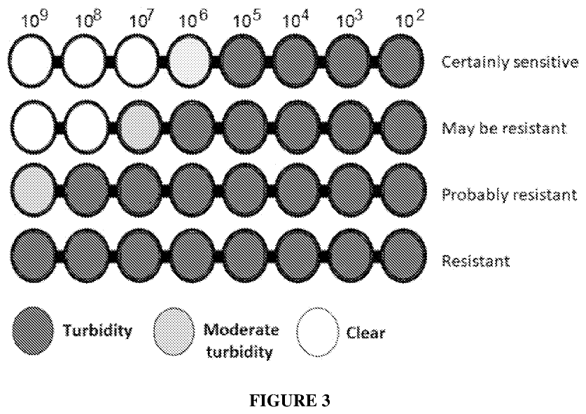

100 μl of suitable culture media is pipetted into wells of a microtitration plate and the phages are left to dissolve. An isolated culture of the bacteria S. aureus is inoculated into 10 ml of the media and grown at 37° C. for 18-24 hours. An inoculum for testing of biological activity of the phages is prepared from the grown culture. The night culture is diluted to the turbidity value of 1 McFarland and 2 ml of this dilution are supplemented to 20 ml of the media/physiological solution. This suspension is poured onto a sterile Petri dish. Sterile tips of a multi-channel pipette are dipped into the suspension and after the dipping, they are transferred and wetted in the wells of a microtitration plate with the dissolved lyophilizate. The plate is cultivated at 37° C. for 12-18 hours under moderate shaking to ensure aeration of the culture. The result is then evaluated based on the turbidity in individual test tubes (FIG. 3). To ensure preciseness of the results, the testing of biological activity should be carried out in 3 parallel experiments.

### Example 2

An isolated culture of the bacteria S. aureus is inoculated into 10 ml of the media and grown at 37° C. for 18-24 hours. 100 μl of suitable culture media is pipetted into wells of a microtitration plate and the phages are left to dissolve. An inoculum for testing of biological activity of the phages is prepared from the grown bacterial culture. 5 μl of the night culture is added to 5 ml of the media (or physiological solution). The volume of 100 μl of this suspension is then transferred into the wells of a microtitration plate comprising dissolved lyophilized phages. The plate is cultivated at 37° C. for 12-18 hours. The result is then evaluated based on the turbidity in individual wells (FIG. 3). To ensure preciseness of the results, the testing of biological effectiveness should be carried out in 3 parallel experiments.

### Example 3

An isolated culture of the bacteria S. aureus is inoculated into 10 ml of the media and grown at 37° C. until the turbidity of 0.5 McFarland. An inoculum for testing of biological activity of the phages is prepared from the grown culture. The volume of 2 ml of this dilution is supplemented to 20 ml of the media/physiological solution. This suspension is poured onto a sterile Petri dish. Sterile tips of a multi-channel pipette are dipped into the suspension and after the dipping, they are transferred and wetted in the wells of the microtitration plate. Before the bacterial culture is prepared, lyophilizate must be dissolved in the wells of the microtitration plate in 100 μl of suitable media. The plate is cultivated at 37° C. for 12-18 hours. The result is then evaluated based on the turbidity in individual test tubes (FIG. 3). To ensure preciseness of results, the testing of biological activity should be carried out in 3 parallel experiments.

### INDUSTRIAL APPLICABILITY

A plate with various types of phages (Table 1) will be stored in a refrigerator and in case an antibiotic treatment of a patient is ineffective, the bacterial strain will be tested for sensitivity to bacteriophages directly in the facility that has isolated and identified the bacteria.

### REFERENCES

Anderson, B., Rashid, M. H., Carter, C., Pasternack, G., Rajanna, C., Revazishvili, T., Dean, T., Senecal, A., & Sulakvelidze, A. (2011). Enumeration of bacteriophage particles. Bacteriophage. https://doi.org/10.4161/bact.1.2.15456.
Andrews, J. M. (2002). Determination of minimum inhibitory concentrations. Journal of Antimicrobial Chemotherapy. https://doi.org/10.1093/jac/dkf083.
Duval, R. E., Grare, M., & Demoré, B. (2019). Fight against antimicrobial resistance: We always need new antibacterials but for right bacteria. In Molecules. https://doi.org/10.3390/molecules24173152.
Lin, D. M., Koskella, B., & Lin, H. C. (2017). Phage therapy: An alternative to antibiotics in the age of multi-drug resistance. World Journal of Gastrointestinal Pharmacology and Therapeutics. https://doi.org/10.4292/wjgpt.v8.i3.162.
Merabishvili, M., Vandenheuvel, D., Kropinski, A. M., Mast, J., De Vos, D., Verbeken, G., Noben, J. P., Lavigne, R., Vaneechoutte, M., & Pirnay, J. P. (2014). Characterization of newly isolated lytic bacteriophages active against Acinetobacter baumannii. PLoS ONE. https://doi.org/10.1371/journal.pone.0104853.

1. A lyophilized Staphylococcus aureus (S. aureus) phage test kit for determination of biological antimicrobial activity of a phage lysate, comprising:
a microtitration plate comprising individual wells of one or more series of wells, wherein each series of wells comprises a strain of lyophilized S. aureus phage lysate at a concentration differing by one order from 1×10⁹ to 1×10²,
wherein the phage is selected from the group consisting of MB401, MB402, MB403, MBSA2, MBSA3, and MBSA5, deposited at the Leibniz Institute DSMZ, German Collection of Microorganisms and Cell Cultures GmbH under the accession numbers DSM 33472, DSM 33473, DSM 33474, DSM 33475, DSM 33476, and DSM 33477, and combinations thereof.

2. The test kit according to claim 1, further comprising CaCl₂ at concentrations of 0.1-1 mM in the individual wells of the microtitration plate.

3. The test kit according to claim 1, further comprising the phage lysate in unit dilutions within one order at concentrations of 1×10⁹-9×10⁹, 1×10⁸-9×10⁸, 1×10⁷-9×10⁷, 1×10⁶-9×10⁶, 1×10⁵-9×10⁵, 1×10⁴-9×10⁴, 1×10³-9×10³, and 1×10²-9×10² PFU/ml.

4. The test kit according to claim 1, which is stable at a temperature of 0-25° C.

5. The test kit according to claim 1, further comprising a lyopholized Staphylococcus aureus bacteria sensitive to the strain of the S. aureus phage.

6. A method of determining biological antimicrobial activity of a Staphylococcus aureus (S. aureus) phage lysate, comprising:
a) adding microbial culture media to the individual wells of the one or more series of wells of the microtitration plate of the lyophilized test kit of claim 1 to dissolve the lyophilized phage lysate,
b) inoculating the dissolved phage lysate with an isolated test sample of S. aureus bacteria, and
c) evaluating the sensitivity of the inoculated test sample of S. aureus bacteria with the naked eye on the basis of the turbidity, wherein a clear test sample inoculated with the S. aureus phage lysate at a concentration on the order of 10⁷ indicates sensitivity of the test sample of S. aureus bacteria to the phage and a turbid test sample inoculated with the S. aureus phage lysate at concentrations of 1×10⁹ to 1×10² indicates resistance of the test sample of S. aureus bacteria to the phage.

7. The test kit according to claim 2, which is stable at a temperature of 0-25° C.

8. The test kit according to claim 2, further comprising a lyopholized Staphylococcus aureus bacteria sensitive to the strain of the S. aureus phage.
